(19) United States
(12) Patent Application Publication
(10) Pub. No.: US 2003/0017607 A1
(43) Pub. Date: Jan. 23, 2003

(54) Detection reaction for epoxides

(76) Inventor: Matthias Boll, Koln (DE)

Correspondence Address: BAYER CORPORATION, PATENT DEPARTMENT, 100 BAYER ROAD, PITTSBURGH, PA, 15205, US

(21) Appl. No.: 10/197,047

(22) Filed: Jul. 16, 2002

(30) Foreign Application Priority Data
Jul. 19, 2001 (DE) 10134431.7

(51) Int. Cl. G01N 33/00

(52) U.S. Cl. 436/128; 436/139; 436/181

(57) ABSTRACT

The present invention relates to a simple method for optical detection of the epoxide and/or aldehyde concentration in the waste gases of chemical reactions, particularly propene oxide and/or acrolein, for example, in the catalyzed partial oxidation of propene. The concentration of the epoxide/aldehyde may be estimated quantitatively by visual means by comparison with a standard or evaluated by digitalization of the image obtained on the detection plate. The detection medium employed in this method is a substrate to which an acid salt has been applied and, optionally, heat treated.

### BACKGROUND OF THE INVENTION

The present invention relates to a simple method for the optical detection of the epoxide and/or aldehyde concentration in the waste gases of chemical reactions, more particularly of propene oxide and/or acrolein, for example, in the catalyzed partial oxidation of propene. The concentration of the epoxide/aldehyde may be estimated quantitatively by visual means or evaluated by digitalization of the image obtained on the detection plate.

Interest in the economic preparation of propylene oxide and acrolein is very great because these materials are starting products for a plurality of other important products. Many of the desired catalysts and/or processes for the preparation of these two starting compounds (such as, for example, the partial direct oxidation of propene) must be operated or carried out at high temperatures so that the detection of the products formed may be carried out mainly on a test scale only by complicated gas chromatographic or comparable methods. The optical detection systems used hitherto (substantially used for ethylene oxide) cannot be applied under the high temperature reaction conditions described above because they are neither sufficiently heat resistant nor sufficiently resistant to oxidation. Due to this restriction in the detection method, the number of catalysts or process parameters which can be examined on a laboratory scale is so severely limited that the probability of developing a favorable new method is low.

No methods for the detection of propylene oxide and acrolein in hot and oxidative waste gases of chemical reactors which could be evaluated in a simple manner have been described in the literature.

The systems employed hitherto used organic reagents which lead either to a color change of the system or to a change in the fluorescence behavior.

Known detection systems are based on organic compounds such as 4-(p-nitrobenzyl)pyridine which changes with ethylene oxide from colorless to blue (Anal. Chem. 27,1435 (1955) and 33, 906 (1961) and J. Pharm. Sci. volume 55, pages 57 and following pages (1961)) and also shows this reaction with propylene oxide. This color reaction is used, for example, in U.S. Pat. No. 4,436,819 for the detection of ethylene oxide in a detection cassette. Various organic detection systems also exist for acrolein, for example, resorcinol or hexyl resorcinol (Z. Lebensm.—Untersuch. u.-Forsch., 99, 352-61 (1954)). Other organic reagent systems which are based on a color change of the detection layer on contact with ethylene oxide are described, for example, in U.S. Pat. No. 2,998,306, U.S. Pat. No. 3,000,706, U.S. Pat. No. 3,258,312, U.S. Pat. No. 3,627,469, U.S. Pat. No. 3,667,917 and U.S. Pat. No. 4,015,937.

Inorganic detection systems are not described. No detection systems which are expected to function sufficiently at elevated temperatures (that is, around 200 degrees Celsius and above) are described either. Similarly, no detection systems have been described as yet which permit highly resolved, quantitative detection of propylene oxide and/or acrolein.

### SUMMARY OF THE INVENTION

The object of the present invention was to provide a method for the detection of propene oxide/acrolein which, on the one hand, may be detected optically in a simple manner and may be used at high temperatures, and on the other hand permits highly resolved detection without cross-sensitivities occurring with C2/C3 acids or neutral substances which could possibly be present in the waste gas.

This and other objects which will be apparent to those skilled in the art are achieved by a method for the detection of epoxidized hydrocarbons and/or aldehydes in which an acid salt is used as a detection medium. This acid salt is applied to a substrate and then optionally, the detection medium is heat-treated in a further step at temperatures above 70° C.

### BRIEF DESCRIPTION OF THE DRAWINGS

FIG. 1 illustrates a detection plate within the scope of the present invention on which the presence of propene oxide and acrolein is indicated as spots.

FIG. 2 is a schematic illustration of a perforated plate with a detection plate within the scope of the present invention positioned behind that perforated plate.

FIG. 3 shows the result of a test for area-resolved detection of propene oxide from Example 2.

FIG. 4 illustrates a detection layer treated with a calibration gas.

FIG. 5 illustrates a section from a detection layer treated with a calibration gas at 200° C.

FIG. 6 illustrates a detection layer treated with acrolein and propylene oxide at 80° C.

FIG. 7 illustrates a detection layer treated with acrolein and propylene oxide at 120° C.

### DETAILED DESCRIPTION OF THE INVENTION

The present invention relates to a method for detecting the presence of epoxidized hydrocarbons and/or aldehydes in which an acid salt is used as the detection medium.

In principle, any epoxidized hydrocarbons may be detected by the method of the present invention. Examples of suitable, detectable epoxidized hydrocarbons include ethene oxide, propene oxide, butene oxide, pentene oxide and the higher homologues thereof. Ethene oxide, propene oxide and butene oxide are preferred.

In principle, any aldehyde may be detected by the method of the present invention. Examples of suitable, detectable aldehydes include unsaturated aldehydes such as acrolein.

The method is used preferably for the detection of epoxidized hydrocarbons and/or aldehydes in gases, particularly preferably in hot and oxidative waste gases of chemical reactors where, in addition to the substances to be detected, a plurality of different chemical compounds, particularly organic acids and organic neutral substances frequently occur.

The term acid salt as used herein means any inorganic salt which contains acid protons in its structure. Acid salts of metals of Groups 1-14 of the Periodic System of Elements according to IUPAC such as lithium, sodium, potassium, magnesium, calcium, iron and lead salts are particularly suitable. Suitable anions include, in particular, hydrogen sulfates, hydrogen sulfites and dihydrogen phosphates.

Lithium, sodium, potassium, magnesium and calcium hydrogen sulfates are particularly preferred.

Suitable substrates include, in particular, porous, heat resistant materials such as plates, spheres, rings, zeolites or other molded bodies, sintered materials such as frits, particularly glass frits, but also calcined clays, silica, and molded bodies made of metal oxides or mixtures thereof.

Coatable, preferably heat resistant fabrics such as those made of asbestos, bentonite, metal, polymer or other fibers which are coated with the detection material are also suitable.

Moreover, coatable materials such as plates, spheres, rings, tubes or other molded bodies made, for example, of glass, polymer or metal which are coated with the detection material are also suitable.

In addition, materials such as those used in high-speed screening methods are also suitable. Examples of such materials include spot plates, racks, silicon plates/wafers and microtiter plates.

If porous or absorbent materials are used, these are advantageously impregnated with a preferably aqueous solution of the acid salt and then dried, partially or completely.

An aqueous solution containing from about 1 to about 30 weight percent of salt is preferably used for impregnation. In order to impregnate the plates as uniformly as possible, it is advantageous to carry out impregnation in an ultrasonic bath. The impregnation process usually takes place within a period of from a few seconds to several days, preferably within minutes. The impregnated plate is advantageously heated to the waste gas temperature prior to use in detecting the epoxidized hydrocarbon or aldehyde.

If detection is carried out at low temperatures (i.e., below 50° C.), the detection layer is usually thermally developed afterwards. This means that the detection layer is heated and kept at an elevated temperature above 50° C. for a period in the range from a few seconds to several days, particularly in the range of minutes. As a rule, the heat treatment takes place at a temperature in the range of from 70° C. to 500° C., particularly advantageously in the range from 80° C. to 200° C. The optimum temperature and period may be determined by a few preliminary tests.

The selectivity, too, can often be influenced by temperature and development time. It is thus easily possible to distinguish, for example, between propene oxide and acrolein by the method of the present invention.

Lithium hydrogen sulfate detection layers brought into contact with acrolein are advantageously developed in the range from 50° C. to 80° C. in the range from 1 to 5 minutes. In this case, the colorless detection layer turns dark to black in the places which came into contact with acrolein.

Lithium hydrogen sulfate detection layers brought into contact with propene oxide are advantageously developed in the range from 100° C. to 180° C. in the range from 5 to 20 minutes. In this case, the colorless detection layer turns dark to black in the places which came into contact with propene oxide.

The development step may usually be omitted if the detection/exposure of the detection layer has already taken place at a correspondingly elevated temperature.

A significant advantage of the method according to the invention is that the method may also be used at temperatures above 200° C. Because the degree of discoloration is often proportional to the epoxide and/or aldehyde concentration, oxidation processes taking place above 180° C. can be monitored on line and, above all, often quantitatively up to a certain limit. The discoloration of the detection layer usually intensifies with increasing exposure. For example, propene oxide and acrolein may usually be detected quantitatively up to a concentration of at least 30 ppm over two hours without cross-sensitivities occurring with C2/C3 acids or neutral substances that could possibly be present in the waste gas. The method permits high-sensitivity resolution of the exposure at the same time.

The method may be combined advantageously with an automated optical evaluation system using an optical detector, digitalization of the image and computerized evaluation of the detected image, as described, for example, in U.S. Pat. No. 6,157,449, and Published Applications PCT/US99/20380 (WO-00/14529-A1) and PCT/US97/18521 (WO98/15805-A1), the teachings of which are incorporated by reference.

The method of the present invention may also be used advantageously in combination with an arrangement of small reactors (for example, a microtiter plate). The reaction gas is brought into contact with various catalysts in the microreactors being tested for epoxides and/or aldehydes by the method of the present invention. To this end, the arrangement of small reactors is operated in close spatial contact with the detection layer.

Having thus described the invention, the following Examples are given as being illustrative thereof.

### EXAMPLES

### Example 1

Distinguishing liquid propene oxide and/or acrolein from other liquid C2 and C3 molecules

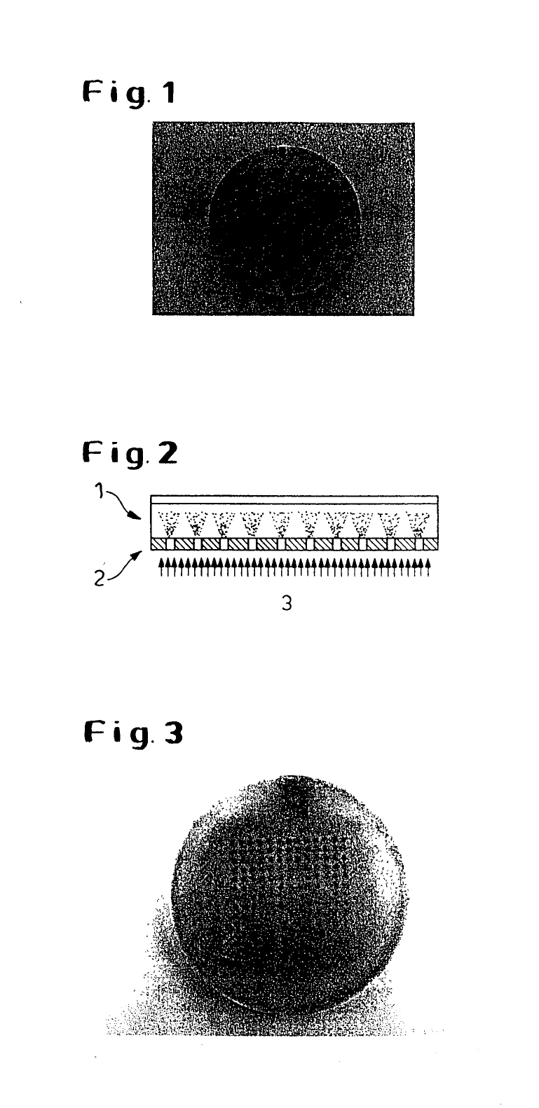

10 μl of propene oxide, acrolein and a mixture of acetaldehyde, propionaldehyde, 2-propanol, acetone, allyl alcohol, propionic acid, 1-propanol and propane 1,2-diol were placed on a detection plate which was prepared by impregnating a glass frit of porosity D4 and having a diameter of 40 mm with 100 ml of a 10 wt. % solution of LiHSO₄ followed by drying at 100° C. for 1 hour, and dried in the air at ambient temperature. The detection plate was then heated for 10 min to 180 degrees Celsius. Propene oxide and acrolein left black spots but the secondary products did not. See FIG. 1 in which the top left indicates acrolein, the bottom center section indicates propene oxide, and the top right section indicates a mixture of secondary product components.

### Example 2

Detection of gaseous propene oxide (978 ppm) in nitrogen (about 98%) carbon monoxide (about 1%) and propene (about 1.01%) at room temperature.

Over a period of 2 hours, a stream of gas having the above-described composition was passed through a perforated plate with a detection plate lying directly behind it. The detection plate was prepared in the same manner as the detection plate used in Example 1. FIG. 2 shows the schematic structure of the apparatus used in this Example. In FIG. 2, the reference number 1 identifies the detection layer on a glass frit impregnated with LiHSO₄, the reference number 2 identifies the perforated plate and reference number 3 identifies the gas containing propene oxide which was analyzed. The composition was confirmed by gas chromatography. The gas mixture was passed through the circular detection layer, which had a diameter of 40 mm, at a mass flow rate of 40 ml per minute at room temperature. The “development” then took place at 180 degrees Celsius over a period of 10 minutes in air. The result is shown in FIG. 3 in which the test for area-resolved detection of propene oxide result was 1000 ppm. The black spot in the upper region of FIG. 3 is an additional mark which was added after the test.

### Example 3

Detection of gaseous propene oxide (33 ppm) in nitrogen (25%) and propene (about 75%) at room temperature.

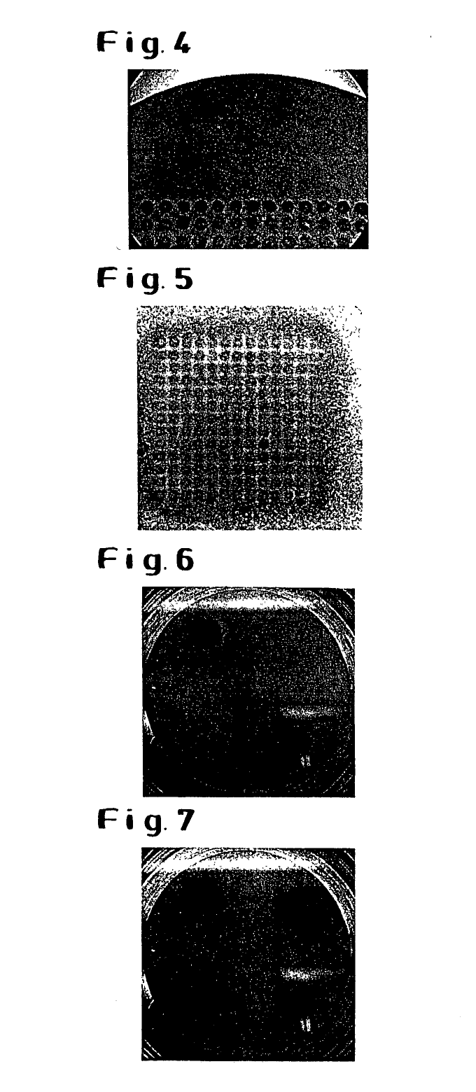

Over a period of 6 hours, a stream of gas having the above-specified composition was passed through a perforated plate with a detection plate lying directly behind it which was prepared in the same manner as in Example 1. (See schematic structure in FIG. 2). The gas mixture was passed through the circular detection layer, which had a diameter of 40 mm, at a mass flow rate of 40 ml per minute at room temperature. The “development” then took place at 180 degrees Celsius over a period of 10 minutes in air. The result is shown in FIG. 4 in which a section of detection layer was treated with a calibration gas with 33 ppm propene oxide at room temperature. The black spot in the upper section of FIG. 4 is a control spot.

### Example 4

Detection of gaseous propene oxide (978 ppm) in nitrogen (about 98%), carbon monoxide (about 1%) and propene (about 1.01%) at 200 degrees.

Over a period of 2 hours and at a detection layer temperature of 200 degrees Celsius, a stream of gas having the above-described composition was passed through a perforated plate with a detection plate lying directly behind it which was prepared in the same manner as the apparatus in Example 1. (Schematic structure shown in FIG. 2.) The gas mixture was passed through the circular detection layer, which had a diameter of 40 mm, at a mass flow rate of 40.1 ml per minute. The “development” then took place at 180 degrees Celsius over a period of 10 minutes in air. A section of the detection layer after development is shown in FIG. 5. A section of the detection layer was treated with a calibration gas with 978 ppm of propene oxide at 200 degrees Celsius.

### Example 5

Distinguishing between acrolein and propene oxide at exposure temperatures below 40 degrees Celsius.

Acrolein and propene oxide were applied as droplets in liquid form to a detection plate which was prepared in the same manner as that used in Example 1. The detection layer was heated slowly after drying in the air, and at about 80 degrees Celsius, the places exposed with acrolein became apparent from the black color. As the temperature continued to increase, the places which were exposed with propene oxide also turned a dark color. FIG. 6 shows the section from a detection layer which was treated with acrolein (top left and bottom right) and propylene oxide (top right and bottom left) at 80° C. FIG. 7 shows the section from a detection layer which was treated with acrolein (top left and bottom right) and propylene oxide (top right and bottom left)) at 120° C.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A method for detecting an epoxidized hydrocarbon and/or aldehyde comprising contacting a gas which could contain such epoxidized hydrocarbon and/or aldehyde with a detection medium produced by
a) applying an acid salt to a substrate, and
b) optionally, heat-treating the substrate from step a) at a temperature above 70° C.

2. The method of claim 1 in which the detection medium is produced from a substrate which is a porous material.

3. The method of claim 2 in which the detection medium is produced with a hydrogen sulfate of an alkaline earth metal or an alkali metal.

4. The method of claim 1 in which the detection medium is produced with a hydrogen sulfate of an alkaline earth metal or an alkali metal.

5. The method of claim 1 in which the detection medium has been heat-treated at a temperature of from 70° C. to 500° C.

6. The method of claim 1 in which acrolein and/or propene oxide are the materials to be detected.
